(19) United States
(12) Patent Application Publication
(10) Pub. No.: US 2022/0404372 A1
(43) Pub. Date: Dec. 22, 2022

(54) Biomarker For Diagnosing Johne's Disease Comprising Alpha-2-Macroglobulin And Uses Thereof

(71) Applicants: SEOUL NATIONAL UNIVERSITY R&DB FOUNDATION, Seoul (KR); INDUSTRY-ACADEMIC COOPERATION FOUNDATION GYEONGSANG NATIONAL UNIVERSITY, Gyeongsangnam-do (KR)

(72) Inventors: Han-Sang YOO, Gyeonggi-do (KR); Min-Kyoung SHIN, Gyeongsangnam-do (KR); Hyun-Eui PARK, Gyeongsangnam-do (KR); Hong-Tae PARK, Gyeonggi-do (KR)

(21) Appl. No.: 17/729,102

(22) Filed: Apr. 26, 2022

(30) Foreign Application Priority Data
Jun. 11, 2021 (KR) 10-2021-0075890

(51) Int. Cl. G01N 33/68; C12Q 1/6883

(52) U.S. Cl. CPC G01N 33/6893; C12Q 1/6883; G01N 2800/065

(57) ABSTRACT

The present invention relates to a biomarker composition for diagnosing Johne's disease using the measurement of alpha-2-macrogolublin (A2M) and use thereof.

Since the biomarker of the present invention can provide improved sensitivity to subclinical infections by revealing differences in the expressions of host proteins in the serum of MAP-infected subjects during various stages of JD progression, it can be effectively used to eradicate JD from a population of subjects. In particular, since the biomarker of the present invention is detected using the ELISA method, it is possible to diagnose Johne's disease more efficiently than when other methods such as mass spectrometry are used, and it can be directly applied in the field. In addition, it is possible to provide a more excellent diagnostic effect than the existing ELISA kits for diagnosing Johne's disease that are commercially available.

### CROSS-REFERENCE TO RELATED APPLICATION

This application claims priority to and the benefit of Korean Patent Application No. 10-2021-0075890, filed on Jun. 11, 2021, the disclosure of which is incorporated herein by reference in its entirety.

### TECHNICAL FIELD

The present invention relates to a biomarker composition for diagnosing Johne's disease using the measurement of alpha-2-macrogolublin (A2M) and use thereof.

### BACKGROUND ART

Johne's disease (JD), which causes significant economic damage to dairy farming worldwide, is a chronic granulomatous enteritis in ruminants caused by Mycobacterium avium subsp. paratuberculosis (MAP). The main hosts of MAP are domestic ruminants, including cattle, sheep, goats and deer, and cases of infection in non-ruminants have also been discovered. In addition, since evidence has been found that MAP is associated with human autoimmune diseases such as Crohn's disease, multiple sclerosis, type 1 diabetes and rheumatoid arthritis, it is an important disease from a public health perspective.

The prominent clinical features of JD are persistent diarrhea, progressive weight loss, decreased milk production and weakness after a long incubation period. During disease progression, infected cattle excrete MAP through feces, which in turn contaminates water, feed, milk and the surrounding environment. Due to the mycolic acid component included in the cell wall of the bacteria, MAP withstands harsh environmental conditions, including dryness, low pH and high or low temperatures, and MAP can survive for more than a year in grassland and survive for a much longer period of time in water. Ingestion of substances contaminated with MAP is a major route of infection. Therefore, rapid detection and culling of subclinical fecal shedders on farms are required for the control of JD.

The diagnosis of JD is broadly divided into two categories. The presence of MAP in clinical specimens such as feces, milk, colostrum and intestinal tissue can be confirmed by PCR and bacterial culture. Bacterial culture using fecal samples from infected animals is the standard for the diagnosis of JD and is the only method for directly detecting live MAP. However, observation of visible colonies requires at least 4 weeks after inoculation due to the slow growth rate. In particular, since subclinical infected individuals show different disease progression rates, the excretion of the bacteria through feces may occur intermittently, resulting in false-negative results for infected animals. Detection of MAP-specific genes such as IS900, ISMap02 and f57 through PCR is rapid, and it is possible to process a relatively large number of samples at once. Nevertheless, PCR-based detection has several limitations. First, various PCR inhibitory components in the fecal sample may interfere with the reaction, resulting in false-negative results. Second, non-specific amplification of host and other bacteria-derived DNA can lead to false-positive results. Third, since most subclinical infected animals usually excrete MAP intermittently, false-negative results may be obtained. The previously developed enzyme-linked immunosorbent assay (ELISA) for MAP diagnosis is a method that can identify MAP-specific antibodies in serum, and it is possible to process a large number of samples at once. However, since most subclinical infected individuals do not have MAP-specific antibodies, false-negative results may be obtained. In addition, co-infection with other pathogens, such as Mycobacterium bovis, Fasciola hepatica and non-tuberculous mycobacteria, may alter the immune response and thereby interfere with accurate diagnosis. In other words, diagnostic techniques that can detect subclinical infected animals are urgently needed for the control and eradication of JD.

Biomarkers are measurable indicators of physiological changes during disease progression. In many studies, in order to identify potential biomarkers in MAP-infected animals, efforts have been made to identify biomarker candidates by analyzing the host transcript in MAP-infected models and discovering several genes that are up-regulated. However, the mRNA expression and protein expression of these genes did not always show a high correlation, and there was a limitation in that it was performed with a relatively small sample size.

### RELATED ART DOCUMENTS

### Non-Patent Documents

(Non-Patent Document 0001) Seo J H, Youn J H, Kim E A, Jun J S, Park J S, Yeom J S, et al. Helicobacter pylori antigens inducing early immune response in infants. J Korean Med Sci. (2017) 32:1139-46. doi: 10.3346/jkms.2017.32.7.1139
(Non-Patent Document 0002) Seth M, Lamont E A, Janagama H K, Widdel A, Vulchanova L, Stabel J R, et al. Biomarker discovery in subclinical mycobacterial infections of cattle. PLoS ONE. (2009) 4:e5478. doi: 10.1371/journal.pone.000547.

### DISCLOSURE

### Technical Problem

Accordingly, as a result of making diligent efforts to provide a JD biomarker for diagnosing MAP-exposed and subclinical MAP-infected cattle by improving the sensitivity of commercial ELISA kits and fecal PCR, which are existing diagnostic methods, the inventors of the present invention completed the present invention by confirming that the serum protein alpha-2-globulin (A2M) can be effectively utilized for the diagnosis of JD in infected animals and furthermore particularly in subclinical cases.

Therefore, it is an object of the present invention to provide a biomarker composition for diagnosing Johne's disease using the measurement of alpha-2-macroglobulin (A2M) and use thereof.

### Technical Solution

The present invention provides a biomarker composition for diagnosing Johne's disease, including an agent for measuring a protein level of alpha-2-macroglobulin (A2M) or an mRNA level thereof.

According to a preferred exemplary embodiment of the present invention, the Johne's disease occurs in a ruminant.

According to a preferred exemplary embodiment of the present invention, the ruminant is cattle, a goat, sheep or a mountain goat.

According to a preferred exemplary embodiment of the present invention, the ruminant includes clinical ruminants and subclinical infected ruminants.

According to a preferred exemplary embodiment of the present invention, the agent for measuring the protein level is at least one selected from the group consisting of an antibody, an interacting protein, a ligand, nanoparticles and an aptamer that specifically bind to the protein or a peptide fragment.

According to a preferred exemplary embodiment of the present invention, the agent for measuring the mRNA level is at least one selected from the group consisting of a primer pair, a probe and an antisense nucleotide that specifically bind to the gene.

In addition, the present invention provides a kit for diagnosing Johne's disease, including the biomarker composition.

According to a preferred exemplary embodiment of the present invention, the kit is a reverse transcription polymerase chain reaction (RT-PCR) kit, a DNA chip kit, an enzyme-linked immunosorbent assay (ELISA) kit, a protein chip kit, a rapid kit or a multiple reaction monitoring (MRM) kit.

In addition, the present invention provides a method for diagnosing Johne's disease, including:
i) measuring a protein level of alpha-2-macroglobulin (A2M) or an mRNA level thereof in a sample isolated from a subject; and
ii) classifying as Johne's disease if the measured level is higher than a normal control level.

According to a preferred exemplary embodiment of the present invention, the sample is at least one selected from the group consisting of blood, plasma, serum, lymph, cerebrospinal fluid, feces, isolated tissue, isolated cells and saliva.

### Advantageous Effects

Since the biomarker of the present invention can provide improved sensitivity to subclinical infections by revealing differences in the expressions of host proteins in the serum of MAP-infected subjects during various stages of JD progression, it can be effectively used to eradicate JD from a population of subjects. In particular, since the biomarker of the present invention is detected using the ELISA method, it is possible to diagnose Johne's disease more efficiently than when other methods such as mass spectrometry are used, and it can be directly applied in the field. In addition, it is possible to provide a more excellent diagnostic effect than the existing ELISA kits for diagnosing Johne's disease that are commercially available.

### DESCRIPTION OF DRAWINGS

FIG. 1A shows representative two-dimensional gel electrophoresis results of bovine serum proteins according to the MAP infection stages in healthy controlgroups. The pI ranges are indicated at the top.

FIG. 1B shows representative two-dimensional gel electrophoresis results of bovine serum proteins according to the MAP infection stages in subclinical shedder groups. The pI ranges are indicated at the top.

FIG. 1C shows representative two-dimensional gel electrophoresis results of bovine serum proteins according to the MAP infection stages in clinical shedder groups. The pI ranges are indicated at the top.

FIG. 2 shows the expression levels of biomarkers (alpha-2-macroglobulin, alpha-1-beta-glycoprotein and transthyretin; (1) to (3)) in bovine serum samples according to the MAP infection stages, and shows the results of detecting bovine serum samples by commercial ELISA diagnostic kits (IDEXX ELISA and IDVet ELISA; (4), (5)). The bovine serum samples were grouped into the following five serum samples from a total of 126 cattle: (a) a healthy control group (HC, n=11): selected from farms without Johne's disease (JD), negative for fecal PCR and negative for ELISA by both commercial kits; (b) an MAP exposure group (E, n=20): selected from JD-positive farms, negative for fecal PCR and negative for ELISA by both commercial kits; (c) a subclinical shedder group (SCS, n=27): positive for fecal PCR and negative for ELISA by both commercial kits; (d) a subclinical non-shedder group (SCNS, n=50): negative for fecal PCR and positive for ELISA by at least one commercial kit; and (e) a clinical shedder group (CS, n=18): positive for fecal PCR and positive for ELISA by at least one commercial kit (* p<0.05; ** p<0.01; *** p<0.001; **** p<0.0001). The optimal cutoff values for A2M, A1BG, TTR, IDEXX and IDVET ELISA are indicated by solid red lines in each graph.

FIG. 3 shows the receiver operating characteristic (ROC) curves of selected biomarkers (A2M, alpha-2-macroglobulin; A1BG, alpha-1-beta glycoprotein; TTR, transthyretin) and two commercial ELISAs (IDEXX and IDVET). The areas under the ROC curves of A2M, A1BG, TTR, IDEXX and IDVET ELISAs were 0.973, 0.641, 0.512, 0.796 and 0.828, respectively, and the optimal cutoff values were 27.56, 11.93, 190.13, 8.92 and 4.37, respectively.

### MODES OF THE INVENTION

Hereinafter, the present invention will be described in more detail.

The term “shedder” of the present invention may refer to an individual which excretes MAP in feces.

The term “clinical ruminant” of the present invention may refer to a ruminant exhibiting the main symptoms of Johne's disease, and the term “subclinical infected ruminant” may refer to a ruminant which is infected with the causative bacteria of Johne's disease such as MAP, but does not show any major symptoms of Johne's disease due to the reasons of the incubation period, the early stage of Johne's disease or the like.

The term “diagnosis” of the present invention means judging the actual condition of an individual's disease in a broad sense in all aspects. The contents of the judgment are the disease name, etiology, disease type, severity, detailed mode of the disease, the presence or absence of complications and the like. In the present invention, diagnosis is preferably to determine the risk of Johne's disease and the onset thereof, which may be caused by infection with the causative bacteria of Johne's disease.

The inventors of the present invention discovered 12 protein biomarkers for JD by using serum samples at different stages of infection. Among the discovered biomarkers, three proteins (A2M, A1BG and TTR) were selected according to their biological significance. A2M is a large glycoprotein involved in capturing a wide range of proteases for host defense. A2M has a usable bait domain for a variety of proteases from pathogens and snare proteases that use structures like ours through a process of structural rearrangement via proteolytic activation. Mycobacterial proteases promote the invasion of host cells, promote growth during infection, and play an important role in a subsequent intracellular survival and disease progression. In addition to inhibiting proteases, A2M also adjusts the biological activity of cytokines and growth factors such as TNF-α, IL-10, IL-2, IL-6, IL-10, bFGF, β-NGF, PDGF and TGF-β. In the early stages of MAP infection, pro-inflammatory cytokines such as IFN-γ, IL-12, TNF-α and IL-6 were significantly increased in MAP-infected animals compared to a healthy control group displaying a Th1-dominant immune response. Conversely, immunological changes occur from Th1 to Th2, and disease progression suggests modulation of the host immune response by pathogens. Therefore, the upregulation of A2M in MAP-infected animals may be associated with the modulation of immune responses during disease progression. A2M is mainly synthesized in the liver and the production thereof is affected by various cytokines such as TNF-α, IL-1 and IL-6. In this regard, the upregulation of A2M may be induced by highly abundant pro-inflammatory cytokines in MAP-infected animals. In the present invention, A2M levels were significantly higher in MAP-infected animals, including both of the exposed and subclinical cases. Therefore, A2M may be a promising biomarker for the differentiation between MAP-infected animals and uninfected animals.

A1BG is a plasma glycoprotein belonging to the immunoglobulin supergene family including an immunoglobulin-like domain. TTR is a tetrameric plasma protein involved in the transport of thyroid hormones and retinol. A1BG was significantly increased only in the exposed group compared to the healthy control group. In addition, elevated serum TTR levels were detected in the subclinical non-shedder group. However, A1BG and TTR could not be utilized as biomarkers for JD, because there was no consistent change between uninfected and infected cattle. This is contrary to a previous study that suggested the upregulation of serum A1BG in cattle experimentally infected with MAP at 12 months (PI) after infection, compared to cattle infected with M. bovis (Non-Patent Document 2). In addition, serum A1BG levels were upregulated in MAP-infected cattle at 3-month PI and simultaneously decreased in uninfected cattle. In addition, experimentally MAP-infected cattle had increased serum TTR abundance at 3 months PI compared to uninfected cattle, and serum TTR levels were consistent up to 10 months compared to uninfected cattle. The difference in the expressions of A1BG and TTR of the present invention may be explained by the origin of the samples. Specifically, in the present invention, serum samples from naturally infected cattle that were at least 1 year old were used, but in [Non-Patent Document 2], since newborn calves that were experimentally infected at 6 weeks of age were used, the immune response was different from that of adult cattle.

In the present invention, it was shown that A2M ELISA yielded higher AUC values and higher sensitivity than two commercial ELISA kits for the detection of subclinical MAP-infected animals. In fact, A2M ELISA showed excellent diagnostic performance for the detection of MAP-infected animals, including subclinical cases. A2M ELISA detected 85.18% of subclinical shedder animals, whereas IDEXX and IDVET ELISA kits detected 0%. In addition, A2M ELISA detected 90% of subclinical non-shedder cases, and the IDEXX and IDVET ELISA kits detected 96% and 52%, respectively. Similarly, A2M ELISA detected 100% of clinical shedder animals, whereas the IDEXX and IDVET ELISA kits detected 94.44% and 88.88%, respectively. In summary, A2M ELISA improved the detection rate of MAP-infected animals, particularly in the subclinical shedder group.

Animals exposed to MAP were negative for both fecal PCR and commercial ELISA kits. However, all exposed animals were co-bred with MAP-infected animals, and some exposed animals were positive for fecal PCR at different time points, suggesting a high likelihood of MAP infection within the exposed group. Serum A2M levels in the MAP-exposed and subclinical shedder groups were significantly higher than in the healthy control group, but were not observed at the serum levels of the other two biomarkers. Moreover, elevated serum A2M levels were consistent in the subclinical shedder group, the subclinical non-shedder group and the clinical shedder group. Therefore, elevated serum A2M levels may be associated with the host immune response to MAP infection at an early stage.

The present invention provides valuable information regarding host serum biomarkers for JD at various stages of infection. In addition, the detailed infection status of each animal, such as histological observations and results of cytokine analysis and tissue bacterial culture, provide more reliable information for statistical analysis.

Accordingly, the present invention may provide a biomarker composition for diagnosing Johne's disease, including an agent for measuring a protein level of alpha-2-macroglobulin (A2M) or an mRNA level thereof.

According to a preferred exemplary embodiment of the present invention, the Johne's disease may occur in a ruminant, and the ruminant may be cattle, a goat, sheep or a mountain goat. Preferably, the ruminant may be cattle.

According to a preferred exemplary embodiment of the present invention, the ruminant may include clinical ruminants and subclinical infected ruminants.

According to a preferred exemplary embodiment of the present invention, the agent for measuring the protein level may be at least one selected from the group consisting of an antibody, an interacting protein, a ligand, nanoparticles and an aptamer that specifically bind to the protein or a peptide fragment.

The term “antibody” refers to a substance that specifically binds to an antigen and causes an antigen-antibody reaction. For the purposes of the present invention, an antibody refers to an antibody that specifically binds to the biomarker for diagnosing John's disease of the present invention. The antibodies of the present invention include all of polyclonal antibodies, monoclonal antibodies and recombinant antibodies. The antibodies may be easily prepared, isolated and purified by using techniques well known in the art. In addition, the antibodies of the present invention include functional fragments of antibody molecules, as well as complete forms having two full-length light chains and two full-length heavy chains. Functional fragments of antibody molecules refer to fragments having at least an antigen-binding function, and for example, there are Fab, F(ab′), F(ab′)2, Fv and the like. In addition, the antibodies of the present invention may be commercially obtained.

The term “aptamer” may be an oligonucleic acid or a peptide molecule.

According to a preferred exemplary embodiment of the present invention, the agent for measuring the mRNA level may be at least one selected from the group consisting of a primer pair, a probe and an antisense nucleotide that specifically bind to the gene.

The term “primer” is a nucleic acid sequence having a short free 3′ hydroxyl group, which may form a complementary template and base pair, and refers to a short nucleic acid sequence that serves as a starting point for template strand copying. Primers are capable of initiating DNA synthesis in the presence of reagents for polymerization (i.e., DNA polymerase or reverse transcriptase) and four different nucleoside triphosphates in appropriate buffers and temperatures. PCR conditions and the length of the sense and antisense primers may be modified based on what is known in the art.

The term “probe” refers to a nucleic acid fragment, such as RNA or DNA, corresponding to several bases for short to several hundred bases for long that are possible to achieve specific binding to a gene or mRNA, and it may be constructed in the form of an oligonucleotide probe, a single-stranded DNA probe, a double-stranded DNA probe, an RNA probe or the like, and may be labeled for easier detection.

The term “antisense” refers to an oligomer having a sequence of nucleotide bases and a backbone between subunits, in which an antisense oligomer is hybridized with a target sequence in RNA by Watson-Crick base pairing, and thereby typically allowing the formation of an mRNA and RNA: oligomeric heteroduplex in a target sequence. An oligomer may have exact sequence complementarity or approximate complementarity to a target sequence.

In addition, the present invention may provide a kit for diagnosing Johne's disease, including the composition.

Since the Johne's disease is identical Johne's disease as a target by the biomarker composition, the description thereof will be replaced with the above description.

According to a preferred exemplary embodiment of the present invention, the kit may be a reverse transcription polymerase chain reaction (RT-PCR) kit, a DNA chip kit, an enzyme-linked immunosorbent assay (ELISA) kit, a protein chip kit, a rapid kit or a multiple reaction monitoring (MRM) kit, and preferably, it may be an enzyme-linked immunosorbent assay (ELISA) kit.

The kit of the present invention may consist of one or more other component compositions, solutions or devices suitable for commonly used methods of analyzing expression levels. For example, a kit for measuring a protein expression level may include a substrate, a suitable buffer, a secondary antibody labeled with a chromogenic enzyme or a fluorescent substance, a chromogenic substrate and the like for the immunological detection of an antibody.

The kit of the present invention may include a sample extraction means for obtaining a sample from the subject to be evaluated. The sample extraction means may include a needle, a syringe or the like. The kit may include a sample collection container for receiving the extracted sample, which may be a liquid, gas or semi-solid. The kit may further include instructions for use. The sample may be any subject sample in which A2M may be present or secreted. For example, the sample may be blood, plasma, serum, lymph, cerebrospinal fluid, feces, isolated tissue, isolated cells and saliva. The measurement of A2M in a subject sample may be performed on whole samples or processed samples.

In addition, the present invention may provide a method for diagnosing Johne's disease, including:
i) measuring a protein level of alpha-2-macroglobulin (A2M) or an mRNA level thereof in a sample isolated from a subject; and
ii) classifying as Johne's disease if the measured level is higher than a normal control level.

According to a preferred exemplary embodiment of the present invention, the sample may be at least one selected from the group consisting of blood, plasma, serum, lymph, cerebrospinal fluid, feces, isolated tissue, isolated cells and saliva, and preferably, it may be serum.

Since the Johne's disease is identical Johne's disease as a target by the biomarker composition, the description thereof will be replaced with the above description.

Hereinafter, the present invention will be described in more detail through examples. These examples are only for illustrating the present invention, and it is apparent to those of ordinary skill in the art that the scope of the present invention is not to be construed as being limited by these examples.

### Example 1

Animal Subjects

Cattle were selected from one farm in Chungcheongnam-do and two farms in Gangwon-do. For the proteomic analysis of serum proteins, 28 cattle were selected according to the results of fecal qPCR analysis and serum MAP antibody levels detected by ELISA and classified into 3 groups as follows: (1) a healthy control group (n=10), selected on farms free of Johne's disease (JD), negative for fecal PCR and serum ELISA; (2) a subclinical shedder group (n=8), positive for fecal PCR and negative for ELISA; and (3) a clinical shedder group (n=10), exhibiting typical JD clinical signs and positive for fecal PCR and ELISA. According to the ELISA results on serum samples detected using two commercial ELISA diagnostic kits (IDEXX Laboratories, Inc., Westbrook, Me., USA; ID Screen Paratuberculosis Indirect, ID Vet, Montpellier, France) for the evaluation of biomarker candidates, 126 cattle were selected, and MAP was detected in stool samples by qPCR targeting IS900 and ISMap02. Specifically, the 126 cattle were divided into 4 groups as follows: (1) a healthy control group (n=11), selected from farms without JD, negative for fecal PCR and negative for ELISA by both commercial kits; (2) an MAP-exposed group (n=20), selected from JD positive farms, negative for fecal PCR and negative for ELISA by both commercial kits; (3) a subclinical shedder group (n=27), positive for stool PCR and negative for ELISA by both commercial kits; (4) a subclinical non-shedder group (n=50), negative for stool PCR and positive for ELISA by at least one commercial kit; and (5) a clinical shedder group (n=18), showing typical JD clinical signs for one or more commercial kits, positive for stool PCR and positive for ELISA. Blood samples were collected by tail vein venipuncture using Vacutainer Plus Plastic Serum Tubes (BD Biosciences, San Jose, Calif., USA). Serum was isolated by centrifugation at 2,500 g for 10 minutes. The isolated serum was transferred to a 1.5 mL tube and stored at −80° C. until use. The animal studies were reviewed and approved by the Animal Ethics Committee of Seoul National University (SNU-200525-4). [Table 1] below shows the characteristics of 28 animals selected for serum profiling. [Table 2] below shows the characteristics of 126 animals selected for the evaluation of biomarker candidates.

TABLE 1

| | | Healthy | Subclinical | Clinical |
|---|---|---|---|---|
| | | control | shedder | shedder |
| | | (n = 10) | (n = 8) | (n = 10) |
| Age, number of | | 2.80 ± 1.31 | 3.50 ± 1.51 | 5.42 ± 1.61 |
| years, mean ± | | | | |
| SD | | | | |
| Gender, female | | 10 (100) | 8 (100) | 10 (100) |
| MAP separa- | | 0 (0) | 1 (12.5) | 8 (80) |
| tion | | | | |
| Fecal PCR | | 0 (0) | 8 (100) | 10 (100) |
| positive | | | | |
| Serum ELISA | IDEXX | 4.06 ± 1.85 | 7.31 ± 4.15 | 235.93 ± 27.67 |
| S/P ratio, | | | | |
| mean ± SD | | | | |

TABLE 2

| | | Healthy | | Subclinical | Subclinical | Clinical |
|---|---|---|---|---|---|---|
| | | control | Exposed | shedder | non-shedder | shedder |
| | | (n = 11) | (n = 20) | (n = 28) | (n = 50) | (n = 18) |
| Age, | | 5.69 ± 1.6 | 4.33 ± 1.6 | 3.83 ± 2.33 | 4.73 ± 1.43 | 5 ± 1.71 |
| number of | | | | | | |
| years, | | | | | | |
| mean ± SD | | | | | | |
| Gender, | | 11 (100) | 20 (100) | 28 (100) | 50 (100) | 18 (100) |
| female | | | | | | |
| MAP | | 0 (0) | 0 (0) | 1 (3.6) | 0 (0) | 14 (77.8) |
| separation | | | | | | |
| Fecal PCR | | 0 (0) | 0 (0) | 28 (100) | 0 (0) | 18 (100) |
| positive | | | | | | |
| Serum | IDEXX | 3.73 ± 1.82 | 8.58 ± 11.74 | 5.84 ±7.79 | 114.21 ± 54.14 | 207.67 ± 102.30 |
| ELISA S/P | | | | | | |
| ratio, mean ± | | | | | | |
| SD | | | | | | |
| | IDVet | 2.49 ± 1.24 | 8.01 ±5.59 | 2.69 ±2.16 | 90.25 ± 77.14 | 183.35 ± 60.99 |

The mean ELISA sample/positive (S/P) ratios for the healthy control group, the subclinical shedder group and the clinical shedder group were 4.16, 7.31 and 235.93, respectively. The mean ages of cattle between the groups did not differ significantly. The mean IDEXX ELISA S/P ratios for the healthy control group, the MAP-exposed group, the subclinical shedder group, the subclinical non-shedder group and the clinical shedder group were 3.73, 8.58, 5.84, 114.14 and 207.67, respectively. Similarly, the mean IDVET ELISA S/P ratios for these groups were 2.49, 8.01, 2.69, 90.25 and 183.35, respectively.

### Example 21

Protein Identification

Through MALDITOF/MS analysis, a total of 12 important regions were identified, and a list of meaningful proteins was generated.

Pooled serum samples were prepared in three groups for the analysis of two-dimensional gel electrophoresis (2-DE) for serum proteins. The Calbiochem ProteoExtract Removal Kit (Merck Millipore, Darmstadt, Germany) was used to remove albumin and IgG from the pooled serum samples according to the manufacturer's instructions. The 2-DE and image analysis using 2-DE samples were performed as previously described (Non-Patent Document 1). The serum protein samples were washed with 40 mmol/L HCl (Tris-hydrochloride, pH 7.2) and 1 mmol/L ethylenediaminetetraacetic acid (EDTA), and dissolved in 9.5 mol/L urea, 4% CHAPS (3-((3-cholamidopropyl)dimethylammonium)-1-propanesulfonate) and 35 mmol/L Tris-HCl (pH 7.2). A rehydration solution including 8 mol/L urea, 4% CHAPS, 10 mmol/L dithiothreitol (DTT) and 0.2% carrier ampholytes (pH 3.0 to 10.0) was mixed with the solubilized protein sample (30 μg) and applied to immobilized pH gradient (IPG) strips (7 cm; Bio-Rad Laboratories, Hercules, Calif., USA) at pH 3.0 to 10.0 on a re-expanded tray (Bio-Rad). Isoelectric focusing (IEF) was performed using protein IEF cells (Bio-Rad), and three pre-set steps included a first conditioning step (15 minutes, 250 Vh), a linear voltage ramping step (3 hours, 4,000 Vh) and a maximum voltage ramping step (up to 30,000 Vh). After IEF, the strips were equilibrated in 0.375 mol/L Tris buffer (pH 8.8) containing 6 mol/L urea, 2% sodium dodecyl sulfate (SDS), 20% glycerol, 2% DTT and 0.01% bromophenol blue. The equilibrated strips were re-equilibrated with the same buffer supplemented with 2.5% iodoacetamide. 2D SDS-PAGE was performed for one day without a stacking gel at 20 mA per gel using a 12.5% detachable polyacrylamide gel (8 to 10 cm).

Protein spots separated from the gel were visualized by silver staining and scanned using the Fluor-S MultiImager (Bio-Rad). The spot intensity of each sample was analyzed using PDQUEST 2D Gel Analysis Software version 6 (Bio-Rad). After silver staining, individual spots were excised from the 2-DE gel and transferred to 1.5 mL tubes. Next, 30 mmol/L potassium ferricyanide and 100 mmol/L sodium thiosulfate were mixed (1:1 ratio), and 100 μL of the mixture was added to the sample and vortexed until the brown color disappeared. Distilled water was added to the sample 3× to stop the reaction. Next, 500 μL of 200 mmol/L ammonium bicarbonate was added to cover the gel for 20 min. The solution was discarded, and the gel pieces were dehydrated with 100 μL of acetonitrile and then dried using vacuum centrifugation. For in-gel digestion, a digestion buffer with 12.5 ng/mL trypsin was added to the gel pieces including the protein spots and incubated on ice for 45 minutes. Next, the enzyme solution was removed and 20 μL of an enzyme-free buffer was added to maintain hydration at 37° C. overnight during the enzymatic reaction. Afterwards, the gel pieces were vigorously vortexed for 30 minutes. The digested solution was transferred to new 1.5 mL tubes and dried using vacuum centrifugation. Finally, the samples were dissolved in 2 μL of 0.1% trifluoroacetic acid (TFA).

For peptide mass fingerprinting, a matrix solution including α-cyano-4-hydroxycinamic acid (40 mg/mL) in 50% acetonitrile and 0.1% TFA was prepared. An equal volume of the matrix solution was added to the sample solution, and 2 μL was transferred to a matrix-assisted laser desorption/ionization time-of-flight (MALDI-TOF)/TOF target plate, dried quickly and washed with deionized water. After drying at room temperature for 10 minutes, the mixed solution was subjected to MALDI-TOF-mass spectrometry (MS) and MS/MS analysis using an ABI 4800 Plus TOF-TOF mass spectrometer (Applied Biosystems, Framingham, Mass., USA). The apparatus was set up for analysis with a 200 Hz Nd:355 nm YAG laser operation. Peaks with a signal/noise ratio of >25 were selected, and the 10 strongest ions were used for MS/MS analysis in 1 kV mode and 1,000 to 1,250 consecutive laser exposures. The Bos taurus protein from the National Center for Biotechnology Information (NCBI) protein database (version 20140415, 2,845 sequences, 921,323 residues) was used for all MALDI-TOF-MS spectral analysis with a molecular weight range of 15%. 2-DE allowed a peptide mass accuracy of 50 ppm. Protein Pilot V.3.0 database software (including MASCOT V.2.3.02 database search engine) was used to analyze MS/MS spectral data at a mass tolerance of 50 ppm. A statistically significant threshold of P=0.05 was used to detect individual peptide ion scores.

TABLE 3

| | | | | | Expected | Increased |
|---|---|---|---|---|---|---|
| Spot No. | Protein name | ID(NCBI) | MW(Da) | p1 | value | group |
| 1 | Complement C3 | A0A4W2D411 | 190190 | 8.36 | 3.20E−04 | Subclinical |
| | | | | | | shedder |
| 2 | Transthyretin | A0A4W2BU20 | 15831 | 5.91 | 2.40E−04 | Subclinical |
| | | | | | | shedder |
| 3 | Apolipoprotein | V6F7X3 | 42963 | 5.3 | 6.70E−24 | Subclinical |
| | A-IV | | | | | and clinical |
| | | | | | | shedders |
| 4 | Alpha-2- | R9QSM8 | 134613 | 5.75 | 1.40E−04 | Subclinical |
| | macroglobulin | | | | | and clinical |
| | | | | | | shedders |
| 5 | IgM heavy | 2232299 | 48512 | 5.68 | 3.10E+02 | Subclinical |
| | chain constant | | | | | and clinical |
| | region | | | | | shedders |
| 6 | Complement | Q693V9 | 34593 | 6.68 | 1.10E−20 | Clinical |
| | component 3d | | | | | shedder |
| 7 | Alpha-1B- | Q2KJF1 | 54091 | 5.29 | 2.40E−05 | Clinical |
| | glycoprotein | | | | | shedder |
| | precursor | | | | | |
| 8 | Kallikrein G | A0A1R3UGP4 | 28249 | 9.07 | 3.40E−02 | Subclinical |
| | | | | | | and clinical |
| | | | | | | shedders |
| 9 | Complement | A0A3Q1MU98 | 58618 | 5.66 | 1.10E−08 | Clinical |
| | component C9 | | | | | shedder |
| | precursor | | | | | |
| 10 | Uncharacterized | A0A3Q1M3L6 | 41077 | 5.16 | 7.90E−04 | Clinical |
| | protein | | | | | shedder |
| 11 | hIgG1 heavy | 7547266 | 36510 | 6.09 | 2.70E−08 | — |
| | chain constant | | | | | |
| | region | | | | | |
| 12 | Inter-alpha- | F1MMD7 | 101620 | 6.22 | 5.00E−03 | — |
| | trypsin inhibitor | | | | | |
| | heavy chain | | | | | |

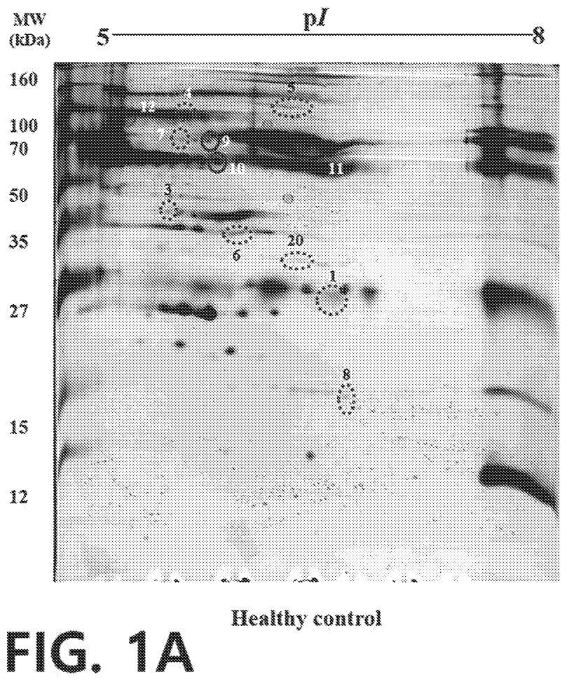

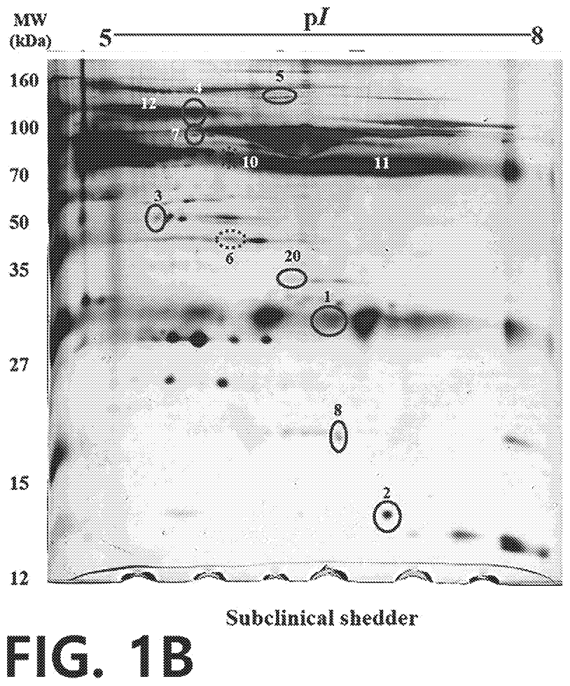

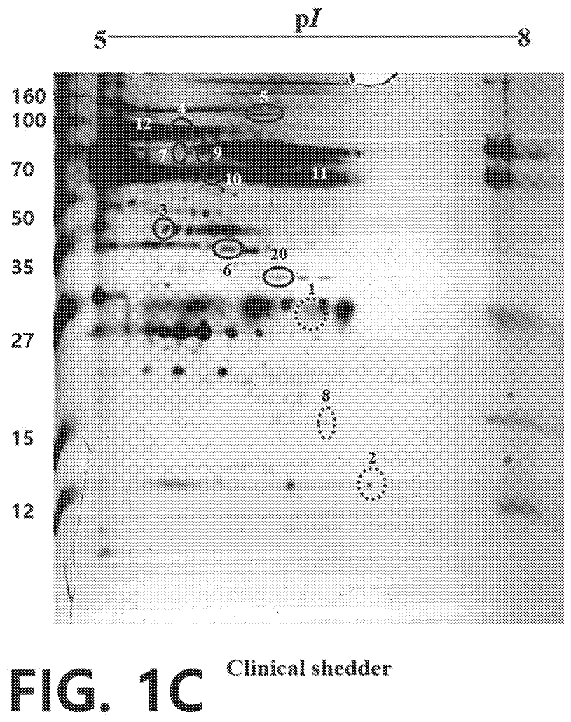

As a result, as shown in [Table 3] above and [FIG. 1A]˜[FIG. 1C], two proteins (complement C3 and transthyretin (TTR)) were abundant only in the subclinical shedder group compared to the healthy control group, and four proteins (complement component 3d, alpha-1B-glycoprotein precursor (A1BG precursor), complement component C9 precursor and uncharacterized protein) were abundant only in the clinical shedder group. In addition, four proteins (apolipoprotein A-IV, alpha-2-macroglobulin (A2M), IgM heavy chain constant region and kallikrein G) were abundant in both of the subclinical shedder group and the clinical shedder group. Identification of the hIgG1 heavy chain constant region or bovine serum albumin suggested the possibility of incomplete clearance of immunoglobin and albumin. A2M, A1BG and TTR were selected for further evaluation of diagnostic performance.

### Example 31

Biomarkers for ELISA

Based on the results of serum ELISA and stool PCR, it was attempted to determine the serum level of each biomarker candidate in different infection groups.

For the quantitative detection of the three selected biomarkers (alpha-2-macroglobulin (A2M), alpha-1-beta glycoprotein (A1BG) and transthyretin (TTR)) in the serum of each animal, a commercially available ELISA kit was used according to the manufacturer's instructions (MyBioSource, San Diego, Calif., USA). The detection ranges of A2M, TTR and A1BG were 0.156 to 10 μg/mL, 312.5 to 5,000 ng/mL and 2.5 to 50 ng/mL, respectively. The intra- and inter-assay CVs of the ELISA kit were <8% and <10%, respectively. Standard curves were generated to determine the concentration of each biomarker in the serum samples.

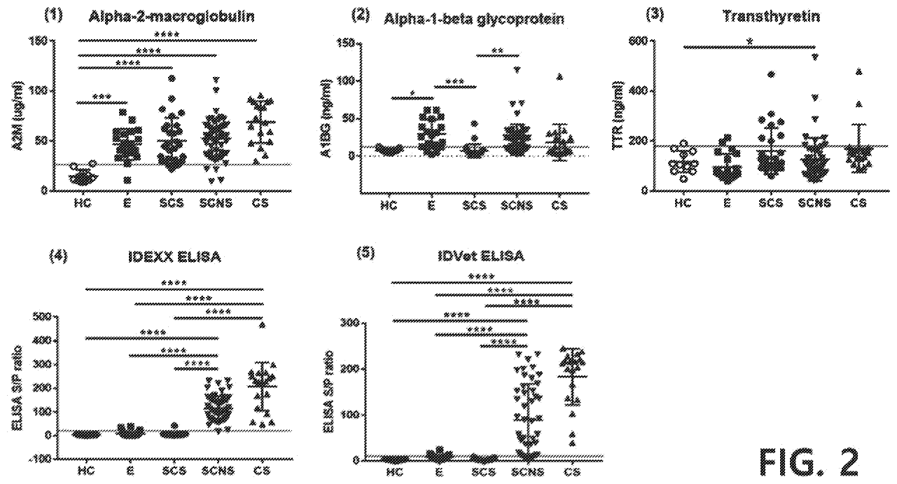

As a result, as shown in [FIG. 2], the serum A2M levels were higher in the MAP-exposed group (E, p<0.01), the subclinical shedder group (SCS, p<0.0001), the subclinical non-shedder group (SCNS, p<0.0001) and the clinical shedder group (CS, p<0.0001) than in the healthy control group. The serum A1BG levels were significantly higher in the MAP-exposed group than in the healthy control group (p<0.05), and significantly lower in the subclinical shedder group than in the MAP-exposed group (p<0.01). In addition, the subclinical shedder group showed higher serum A1BG levels than the subclinical shedder group (p<0.01). The serum TTR levels were significantly higher in the subclinical non-shedder group than in the healthy control group (p<0.05), but there was no significant difference between the other infection groups.

### Example 41

Diagnostic Utility of Selected Biomarkers

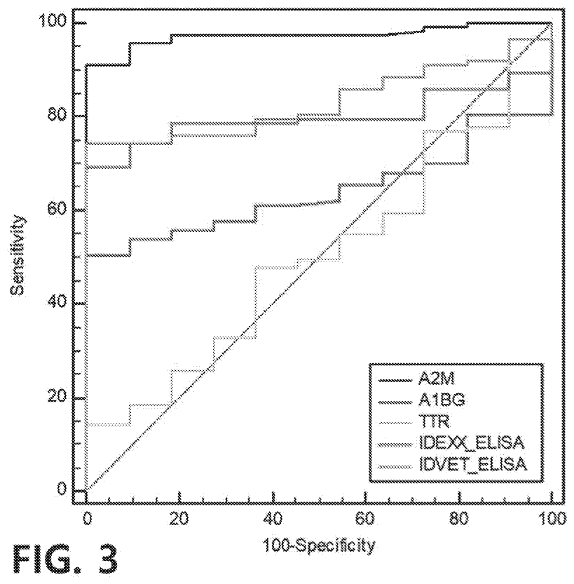

The diagnostic performance of the candidate biomarker proteins compared to commercial ELISA kits was presented by group (Table 4). All subjects in groups 1, 2 and 3 corresponding to clinical shedders were positive for A2M ELISA. In particular, 25 out of 26 animals in groups 5 and 6, which were subclinical non-shedders and tested positive only by one of the commercially available kits, were diagnosed as positive by A2M-ELISA. Above all, in the diagnosis using A2M-ELISA in group 7 belonging to the subclinical shedder and group 8 belonging to the exposed group, 23 out of 27 animals and 18 out of 20 animals were respectively diagnosed as positive (Table 3). The ROC curve analysis suggested the possibility of discrimination between different infection groups using biomarker-based ELISA. The AUC and optimal cutoff values were calculated (FIG. 3). When comparing both of the healthy control group (n=11) and the infected cattle groups (n=115), A2M ELISA showed excellent discrimination ability with AUC=0.973 (95% confidence interval [CI]: 0.946 to 1.000, p<0.0001). The sensitivity was 90.4%, and the specificity was 100%. Conversely, A1BG ELISA showed poor discrimination ability with AUC=0.641 (95% CI: 0.543 to 0.739, p=0.0048), a sensitivity of 50.4%, and a specificity of 100%. Similarly, TTR ELISA showed poor discrimination ability with an AUC value of 0.512 (95% CI: 0.348 to 0.677, p=0.8840), a sensitivity of 15.7% and a specificity of 100%. The IDEXX commercial ELISA kit showed fair discrimination ability with an AUC value of 0.796 (95% CI: 0.720 to 0.872, p<0.0001), a sensitivity of 67.83% and a specificity of 100%. In addition, the IDVET commercial ELISA kit showed excellent identification ability with an AUC value of 0.828 (95% CI: 0.753 to 0.904, p<0.0001), a sensitivity of 73.04%, and a specificity of 100%. When the ROC curves obtained using the three biomarkers were compared with the ROC curves obtained using the two commercial kits, A2M showed excellent diagnostic performance (FIG. 3).

TABLE 4

| | Group | | | | | | | | | IDEXX | | IDVET | |
|---|---|---|---|---|---|---|---|---|---|---|---|---|---|
| | | IDEXX | IDVET | A2M | | A1BG | | TTR | | ELISA | | ELISA | |
| | PCR | ELISA | ELISA | P | N | P | N | P | N | P | N | P | N |
| 1 | P | P | P | 15 | 0 | 7 | 8 | 1 | 14 | 15 | 0 | 15 | 0 |
| 2 | P | P | N | 2 | 0 | 1 | 1 | 0 | 2 | 2 | 0 | 0 | 2 |
| 3 | P | N | P | 1 | 0 | 0 | 1 | 1 | 0 | 0 | 1 | 1 | 0 |
| 4 | N | P | P | 21 | 3 | 17 | 7 | 1 | 23 | 24 | 0 | 24 | 0 |
| 5 | N | P | N | 23 | 1 | 13 | 11 | 3 | 21 | 24 | 0 | 0 | 24 |
| 6 | N | N | P | 2 | 0 | 1 | 1 | 2 | 0 | 0 | 2 | 2 | 0 |
| 7 | P | N | N | 23 | 4 | 2 | 25 | 8 | 19 | 0 | 27 | 0 | 27 |
| 8 | N | N | N | 18 | 2 | 16 | 4 | 2 | 18 | 0 | 20 | 0 | 20 |
| 9 | N | N | N | 0 | 11 | 0 | 11 | 0 | 11 | 0 | 11 | 0 | 11 |

### [Statistical Analysis]

The ANOVA with Tukey's post hoc test between different infection groups was performed using GraphPad Prism software version 7.00 (GraphPad Software, Inc., La Jolla, Calif., USA). P-values less than 0.05 were considered statistically significant. The receiver operating characteristic (ROC) curve analysis was performed to determine the areas under the curve (AUC) and the optimal cutoff values for each biomarker candidate. The ROC curve analysis was performed using MedCalc software version 19.4 (MedCalc Software, Ostend, Belgium). The optimal cutoff values were determined as values representing the maximum Youden Index (J=Se+Sp−1). The ability to discriminate between the different infection groups and the healthy control group was determined with the following meanings. Specifically, biomarkers with AUC values of 0.9 or more were considered to have the most excellent discrimination ability. AUC values of ≥0.8 and <0.9 were considered to have excellent discrimination ability. AUC values of ≥0.7 and <0.8 were considered to have fair discrimination ability. AUC values of <0.7 were considered to have low discrimination ability.

1. A biomarker composition for diagnosing Johne's disease, comprising an agent for measuring a protein level of alpha-2-macroglobulin (A2M) or an mRNA level thereof.

2. The biomarker composition of claim 1, wherein the Johne's disease occurs in a ruminant.

3. The biomarker composition of claim 2, wherein the ruminant is cattle, a goat, a sheep or a mountain goat.

4. The biomarker composition of claim 2, wherein the ruminant includes clinical ruminants and subclinical infected ruminants.

5. The biomarker composition of claim 1, wherein the agent for measuring the protein level is at least one selected from the group consisting of an antibody, an interacting protein, a ligand, nanoparticles and an aptamer that specifically bind to the protein or a peptide fragment.

6. The biomarker composition of claim 1, wherein the agent for measuring the mRNA level is at least one selected from the group consisting of a primer pair, a probe and an antisense nucleotide that specifically bind to the gene.

7. A kit for diagnosing Johne's disease, comprising the biomarker composition of claim 1.

8. The kit of claim 7, wherein the kit is a reverse transcription polymerase chain reaction (RT-PCR) kit, a DNA chip kit, an enzyme-linked immunosorbent assay (ELISA) kit, a protein chip kit, a rapid kit or a multiple reaction monitoring (MRM) kit.

9. A method for diagnosing Johne's disease, comprising:
i) measuring a protein level of alpha-2-macroglobulin (A2M) or an mRNA level thereof in a sample isolated from a subject; and
ii) classifying as Johne's disease if the measured level is higher than a normal control level.

10. The method of claim 9, wherein the sample is at least one selected from the group consisting of blood, plasma, serum, lymph, cerebrospinal fluid, feces, isolated tissue, isolated cells and saliva.
